(19) United States
(12) Patent Application Publication
(10) Pub. No.: US 2006/0203963 A1
(43) Pub. Date: Sep. 14, 2006

(54) Medical apparatus system, and method for operation thereof

(76) Inventors: Helmut Biedermann, Uttenreuth (DE); Petra Popp, Munchen (DE)

Correspondence Address: SCHIFF HARDIN, LLP;PATENT DEPARTMENT, 6600 SEARS TOWER, CHICAGO, IL, 60606-6473, US

(21) Appl. No.: 11/355,131

(22) Filed: Feb. 14, 2006

(30) Foreign Application Priority Data
Feb. 14, 2005 (DE) 10 2005 006 661.5

(51) Int. Cl. G01N 23/04; A61B 5/05

(52) U.S. Cl. 378/62; 600/407

(57) ABSTRACT

In a method for operation of a medical apparatus system having a number of components, namely at least one medical-technical imaging apparatus, at least one computer station, as well as a communication network connecting these components, a parameter setting of the medical-technical apparatus that can be selected for the implementation of an examination is stored, image data are generated with the medical-technical apparatus with the established parameter setting, and the image data are correlated with the parameter setting such that the image data, or the image data with parameter setting, or only the parameter setting can be selectively transmitted over the communication network.

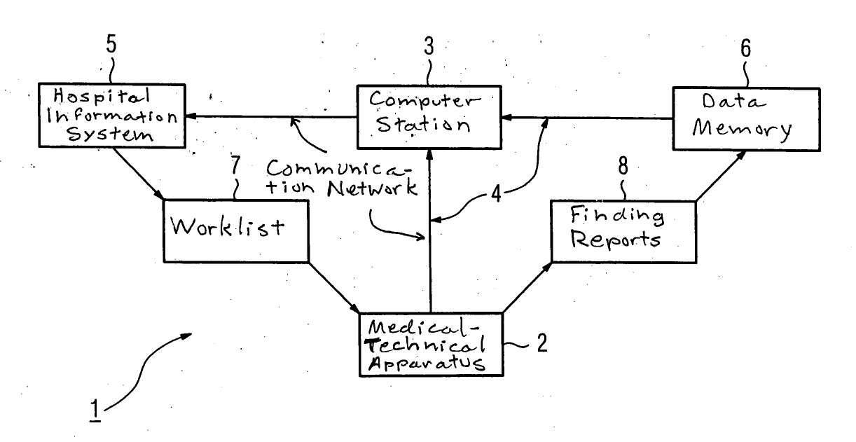

### BACKGROUND OF THE INVENTION

1. Field of the Invention

The present invention concerns a method for operation of a medical apparatus system with at least one imaging medical-technical apparatus, as well as such a system for implementing the method. Such an apparatus system is, for example, known from DE 199 22 793 B4.

2. Description of the Prior Art

Medical apparatus systems are increasingly networked with the aid of a hospital information system (KIS), a radiological information system (RIS) and/or similar systems. An example of such an apparatus system is described in DE 199 22 793 B1. For example, the DICOM (Digital Imaging and Communications in Medicine) standard is used for the transfer of images and further medical information to enable the digital communication between diagnosis and therapy apparatuses.

### SUMMARY OF THE INVENTION

An object of the present invention is to further increase the use of medical apparatus systems of the type having at least one imaging apparatus as well as a communication network for data-technical linking of various components, in particular in a hospital.

The above object is achieved in accordance with the present invention in a medical apparatus system, and a method for operating a medical apparatus system, wherein the system has a number of components including at least one medical imaging apparatus, at least one computer station, and a communication network connecting these components, and wherein a parameter setting, that is selected for operating the medical imaging apparatus to implement an examination, is stored, image data are generated with the medical imaging apparatus during the examination with the established parameter setting, and the image data are correlated with the parameter setting to allow only the image data, or the image data with the parameter setting or only the parameter setting, to be selectively transmitted via the communication network.

As components networked by means of a communication network, the apparatus system has at least one imaging medical-technical apparatus (in particular an x-ray-technical apparatus such as, for example, a computed tomography apparatus) as well as a computer station. Furthermore, the medical apparatus system can be, for example, at least one therapy apparatus, such as an irradiation apparatus. In order to implement an examination with the medical-technical apparatus, parameters must be set at the apparatus. The parameter set is designated dependent on the type of the medical-technical apparatus, for example as a scan protocol. The parameter setting is stored within the medical-technical apparatus system, for example in a central data store. The input of the parameters does not need to occur at the medical-technical apparatus (generally also called a modality). Rather, for example, it is possible for the parameter setting to be conducted in advance by a person (known as an examination planner) given this responsibility. The entry of the parameters can ensue, for example, at the computer station within the medical apparatus system. Such a parameter setting temporally and spatially separate from the diagnosis has the advantage of affording the possibility to check the setting of the parameters again before the examination. Neither the selection of the parameters for the examination nor the monitoring of the parameter setting caused thereby contribute to demands of the available examination time of the medical-technical apparatus. The medical-technical apparatus thus exhibits a very high degree of availability with a simultaneously more comfortable and easily controllable possibility of parameter setting. Adjustment parameters concern, for example, exposure time, exposure dose or a feed speed for an examination series.

Image data are generated with the medical-technical apparatus with the parameter setting established in advance. For example, such data can represent a digitized x-ray exposure or can be raw data from which images can be generated via Fourier transformation. Like the entered parameter setting, the acquired image data are stored in the medical apparatus system. The image data are thereby correlated with the parameter data, such that the relation of the image data to the parameter data can be traced (reproduced) at any time, but also the possibility exists to transmit data over the communication network and to process data concerning the parameters independently of the image data. At every site of the medical apparatus system, the possibility is by provided to view parameter settings that were selected in the operation of the medical-technical apparatus and to change them as needed, in particular before using them again. Parameter settings of various examinations can be automatically stored in a data memory associated with the computer station, or integrated into the computer. Independent of which components within this system the user operates, the user of the medical apparatus system thus always has the possibility to access any parameter settings used in the past for reuse in identical or modified form.

In an embodiment, parameter settings can be automatically optimized by recurring (recursive) comparisons of entered parameter settings with image data acquired therewith. The optimization can either ensue without any intervention of the user or after approval by the user of an automatically proposed changed parameter set.

The DICOM procedure (which, for example, is known in principle from DE 196 25 837 A1) is advantageously used for transfer of a stored parameter setting within the communication network. Parameter settings of the medical-technical apparatus are preferably stored in a DICOM header. Storage in the DICOM-SR (structured report) format has proven to be particularly advantageous.

An advantage of the invention is that an integrated workflow in a hospital is enabled in a simple manner, allowing parameter data of medical-technical apparatuses to be recalled at any node of a communication network and can be reused at the point of recall as well as edited, as necessary. Given connection of the communication network of the hospital to a worldwide data network (in particular the Internet), the stored parameter settings (in particular in scan protocols) are in principle globally available. Data protection requirements can be satisfied by suitable allocation of access rights as well as by encryption technologies.

### DESCRIPTION OF THE DRAWINGS

An exemplary embodiment of the invention is shown in the single FIGURE that illustrates the inventive method for operation of a medical apparatus system.

### DESCRIPTION OF THE PREFERRED EMBODIMENTS

A medical apparatus system 1 has at least one imaging medical apparatus 2, also designated as a modality. A number of modalities (for example a computed tomography apparatus, a magnetic resonance apparatus, a DSA unit for digital subtraction angiography and/or an x-ray unit for digital radiography) can form the medical apparatus system 1. The medical apparatus system 1 furthermore has at least one computer system 3, i.e. a computer including input and output devices. The at least one imaging medical-technical apparatus 2 as well as the at least one computer station 3 are networked by means of a communication network 4 that advantageously exhibits a connection with the World Wide Web.

As components of the apparatus system 1, the medical-technical apparatus 2 as well as the computer station 3 are coupled in a data-technical sense with a hospital information system 5 which represents a further component of the medical apparatus system 1 or is at least coupled with this apparatus system 1. Instead of the hospital information system 5, a radiology information system (RIS) and/or a cardiology information system (CIS) can be provided. The hospital information system (KIS) 5 can likewise include an RIS and/or a CIS. A data memory 6 which can likewise be a part of the hospital information system 5 is also located in the medical apparatus system 1. The separate representation of the computer station 3, the hospital information system 5 and the data memory 6 in the schematic figure is to be understood only as a conceptual division and other physical arrangements of the cited components 3, 5, 6 can be used.

The transfer of information in the communication network 4 can ensue in any manner, wireless or wired. The computer station 3 is spatially separate from the medical-technical apparatus 2, such that a data transfer over a spatial distance actually has to ensue given a communication between these components 2, 3. In order to be able to operate the modality 2 (for example a computer tomography apparatus), i.e. to be able to examine a patient, various parameters must be set. These parameters concern, for example, the position of the patient, settings of the exposure, the spatial acquisition region, the acquisition time as well as settings of the image evaluation. In a conventional manner, the parameter settings would have to be effected directly at the medical-technical apparatus 2. This initially entails the disadvantage that the time required for the parameter setting is no longer available for the examination of a patient by means of the medical-technical apparatus 2. Moreover, the frequent adjustment of parameters at the modality 2 also represents a significant error source. In order to avoid these disadvantages, in the symbolically-represented medical apparatus system 1 the possibility is provided to effect the parameter setting with regard to the medical-technical apparatus 2 at the computer station 3 that is spatially separate therefrom this and is connected merely by means of the communication network 4.

In the simplest case, a specific parameter setting for the medical-technical apparatus 2 can be manually entered on the computer station 3 and be transferred to the apparatus 2 via the communication network 4. The computer station 3 advantageously offers the possibility to access stored data sets of parameter settings which can be stored in the computer station 3 itself, in the hospital information system 5 and/or in the data store 6. Various sets of parameters for different examinations that can be implemented with the apparatus 2 are stored in a logical structure, known as protocol references. Each stored parameter setting thereby corresponds to an actual exposure acquired with the imaging medical-technical apparatus 2. The storage is structured such that the connection between a parameter set and an image data set can be established at any time, but the parameter data set also can be transferred independent of the image data set in the communication network 4 as needed. Parameter data and image data can be physically stored either on a common data medium or on separate data media.

The DICOM standard is particularly suited for the transfer of the image data in the communication network 4. The parameter settings of the medical-technical apparatus 2 belonging to an image can be contained in a DICOM header of the image. It is likewise also possible to merely incorporate a link to a specific parameter setting in the DICOM header and to store the parameter setting itself in a separate file. The latter cited variant in particular has the advantage that extensive information can be associated with an image. It is particularly advantageous to represent the parameters of the apparatus 2 (also known as scan parameters in the case of a computed tomography apparatus as the apparatus 2) in a DICOM-SR (structured report) format. It is thereby possible to convert the typically machine-specific formatting of the parameters into a generally understandable format, in particular into text format. Moreover, it is also possible to standardize a short form (possibly in encrypted form) of parameter data sets independent of the modality. A specific parameter setting thus can be transferred, decrypted and thus displayed to the operator of the medical-technical apparatus 1 in long form as needed, independently of the type of the medical-technical apparatus.

If the active operator at the computer station 3 has selected a suitable parameter setting for an examination with the modality 2, the operator sends the corresponding parameter data to the hospital information system 5 via the communication network 4. The selection and processing of the parameter data is also possible in the same manner at any arbitrary other point of the apparatus system 1. Drawing upon the selected parameter data, the hospital information system 5 generates a worklist 7 in the form of a DICOM worklist. This is transferred to the imaging medical-technical apparatus 2 via the communication network 4. Alternatively, it is possible to directly transfer parameter data to the medical-technical apparatus 2 from the computer station 3.

In any case, after the examination implemented with the apparatus 2 an electronic finding report 8 is generated which is conducted over the communication network 2 to the data store 6, which can be part of the hospital information system 5. The logical connection of the parameter setting used with the finding data is thereby maintained, such that the corresponding data can be used as needed in a simple manner for future examinations with the imaging medical-technical apparatus 2. The hospital information system 5 recurrently evaluates (in a manner not shown) the growing number of finding reports 8 (which in particular comprise image data) in connection with the associated parameter data and therewith automatically generates suggestions for improved, examination-specific parameter settings of the medical-technical apparatus 2.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for operating a medical apparatus system comprising a plurality of components, including at least one medical imaging apparatus, at least one computer station, and a communication network connecting said plurality of components, said method comprising the steps of:
at said computer station allowing selection of a parameter setting for said medical imaging apparatus to implement an examination, and storing said parameter setting;
operating said medical imaging apparatus to generate image data during said examination using said parameter setting; and
correlating said image data with said parameter setting to allow only said image data, or said image data with said parameter setting, or only said parameter setting, to be selectively transmitted via said communication network.

2. A method as claimed in claim 1 comprising selecting different parameter settings for a plurality of different examinations to be implemented with said medical imaging apparatus and automatically storing each of said parameter settings in a data memory associated with said computer station.

3. A method as claimed in claim 2 comprising optimizing said parameter settings by comparing said parameter settings with said image data generated by said medical imaging apparatus using each of said parameter settings.

4. A method as claimed in claim 1 wherein said computer station is spatially separated from said medical imaging apparatus, and comprising transferring said parameter setting from said computer station to said medical imaging apparatus via said communication network.

5. A method as claimed in claim 4 comprising transferring said parameter setting from said computer station to said medical imaging apparatus via said communication network according to the DICOM standard.

6. A method as claimed in claim 5 comprising storing said parameter setting in a DICOM header.

7. A method as claimed in claim 6 comprising storing said parameter setting according to the DICOM-SR format.

8. A medical apparatus system comprising:
a medical imaging apparatus;
a computer station;
a communication network placing said computer station in communication with said medical imaging apparatus;
said computer station allowing entry of a selected parameter setting to operate said medical imaging apparatus to implement an examination, and said computer station effecting storage of said parameter setting;
said medical imaging apparatus be operated with said parameter setting to generate image data in said examination according to said parameter setting; and
said computer station correlating said image data with said parameter setting and allowing selective transmission, via said communication network, of only said image data, or said image data with said parameter setting, or only said parameter setting.

9. A medical apparatus system as claimed in claim 8 wherein said medical imaging apparatus is an x-ray imaging apparatus.
